(19) United States
(12) Patent Application Publication
(10) Pub. No.: US 2012/0058147 A1
(43) Pub. Date: Mar. 8, 2012

(54) Geographically Region Specific Allergy Immunotherapy for Companion Animals

(76) Inventor: Jonathan D. Plant, Lake Oswego, OR (US)

(21) Appl. No.: 12/877,803

(22) Filed: Sep. 8, 2010

(51) Int. Cl. A61K 39/35; A61P 37/08

(52) U.S. Cl. 424/275.1

(57) ABSTRACT

Implementations and techniques for providing geographically region specific allergy immunotherapy for companion animals are generally disclosed.

### BACKGROUND

In common practice, a veterinarian may clinically diagnose a companion animal with atopic dermatitis. To prescribe allergy immunotherapy, the veterinarian may provide allergy testing of the companion animal for a variety of potential allergens and order a mixture including allergens that provide positive test responses. The mixture may be administered to the companion animal to induce allergen tolerance. The allergy immunotherapy may provide the animal relief from pain and discomfort.

### BRIEF DESCRIPTION OF THE DRAWINGS

Embodiments are illustrated by way of example and not by way of limitation in the figures of the accompanying drawings, in which like references may indicate similar elements and in which:

FIG. 1 is an illustration of a flow chart of an example method for providing allergy immunotherapy to a companion animal;

FIG. 2 is an illustration of a flow chart of an example method for defining geographic regions and allergy immunotherapy formulations for companion animals;

FIG. 3 is an illustration of a flow chart of an example method for providing allergy immunotherapy to a companion animal;

FIG. 4 is an illustration of a flow chart of an example method for providing allergy immunotherapy to a companion animal;

FIG. 5 is an illustration of a flow chart of an example method for providing allergy immunotherapy to a companion animal;

FIG. 6 is an illustration of a map of an example area divided into example geographic regions that may correspond to allergy immunotherapy formulations; and

FIGS. 7A to 7H are tables of example regions and corresponding allergy immunotherapy formulations including allergen components.

### DETAILED DESCRIPTION

In the following description, various embodiments will be disclosed. However, it will be apparent to those skilled in the art that the embodiments may be practiced with all or only some of the disclosed subject matter. For purposes of explanation, specific numbers and/or configurations are set forth in order to provide a thorough understanding of the embodiments. However, it will also be apparent to one skilled in the art that the embodiments may be practiced without one or more of the specific details, or with other approaches and/or components. In other instances, well-known structures and/or operations are not shown or described in detail to avoid obscuring the embodiments.

References throughout this specification to embodiments or examples mean that a particular feature, structure, or characteristic described in connection with the embodiment or example is included in at least one embodiment or example. The appearances of references to embodiments or examples in various places throughout this specification are not necessarily referring to the same embodiment or example. Furthermore, the particular features, structures, or characteristics may be combined in any suitable manner in the discussed embodiments and examples.

In various embodiments of the claimed subject matter, methods for providing improved allergy immunotherapy to companion animals are described.

In an embodiment, a veterinarian may make a clinical diagnosis of atopic dermatitis in a companion animal (e.g., a dog, a cat or a horse). Upon diagnosis, a location may be associated with the companion animal. For example, the associated location may be where the animal lives or a location that may be causing the atopic dermatitis. The location may be used to choose a geographic region associated with the companion animal. For example, the associated geographic region may be the geographic region that includes the location. The associated geographic region may be one of several geographic regions that a larger area may have been divided into for the purpose of determining an allergy immunotherapy formulation for the companion animal. For example, an allergy immunotherapy specialist, veterinarian or manufacturer may divide a larger coverage area into regions such that each region has an associated allergy immunotherapy formulation that may be applied to a companion animal from that region.

Thereby, based on the associated geographic region, an allergen immunotherapy formulation may be defined for treatment of the companion animal. The formulation may determine which allergens may be included in an allergen mixture that may be administered to the companion animal. For example, different formulations may be provided for each of the different geographic regions and the formulation of the geographic region associated with the companion animal may be ordered. The mixture based on the formulation may be produced and administered to the companion animal. The administration of the mixture may induce allergen tolerance in the companion animal. The allergy immunotherapy may provide relief to the animal.

The methods discussed herein may provide the advantage of foregoing allergy testing. Allergy testing may be costly, difficult and time consuming. In some examples, the efficacy of allergy testing may be of questionable value.

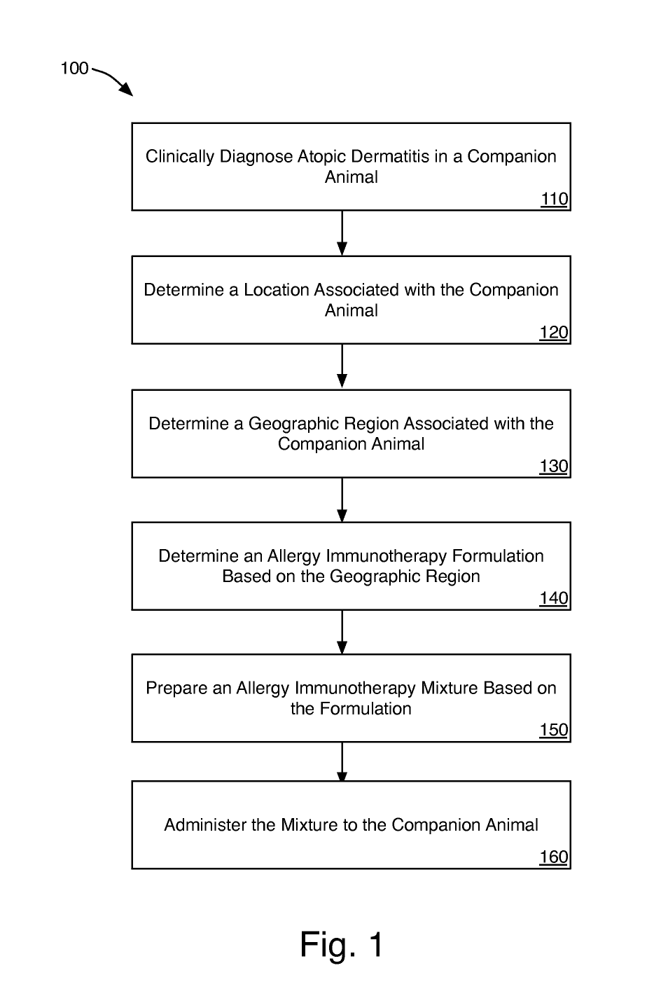

Turning now to FIGS. 1-7, various embodiments will be described. In FIG. 1, a flow chart illustrates a method 100 for providing allergy immunotherapy to a companion animal. At block 110, atopic dermatitis may be clinically diagnosed in a companion animal (e.g., a dog, a cat or a horse). In an example, a veterinarian may diagnose atopic dermatitis in the companion animal. In general, the diagnosis may be made according to typical veterinary protocols. In some examples, the diagnosis may be made based on visual and physical evaluations of the companion animal. The method may continue at block 120.

At block 120, a location may be associated with the companion animal. In various examples, the association may be made by a veterinarian, a veterinarian's assistant, an allergy immunotherapy specialist, or the companion animal's owner or the like. In general, the location associated with the companion animal may be the location where the animal lives or the location that may be causing the animal's atopic dermatitis. In an example, the location associated with the animal may be the address where the animal lives. The method may continue at block 130.

At block 130, a geographic region may be associated with the companion animal. In various examples, the association may be made by a veterinarian, a veterinarian's assistant or the like, an immunotherapy specialist, or an allergen manufacturer, or the like. In an example, the geographic region associated with the companion animal may be a geographic region including the location associated with the geographic animal.

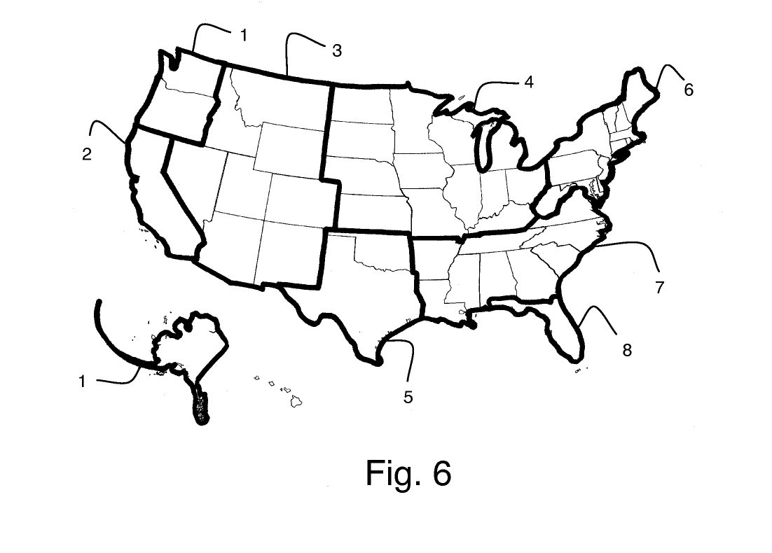

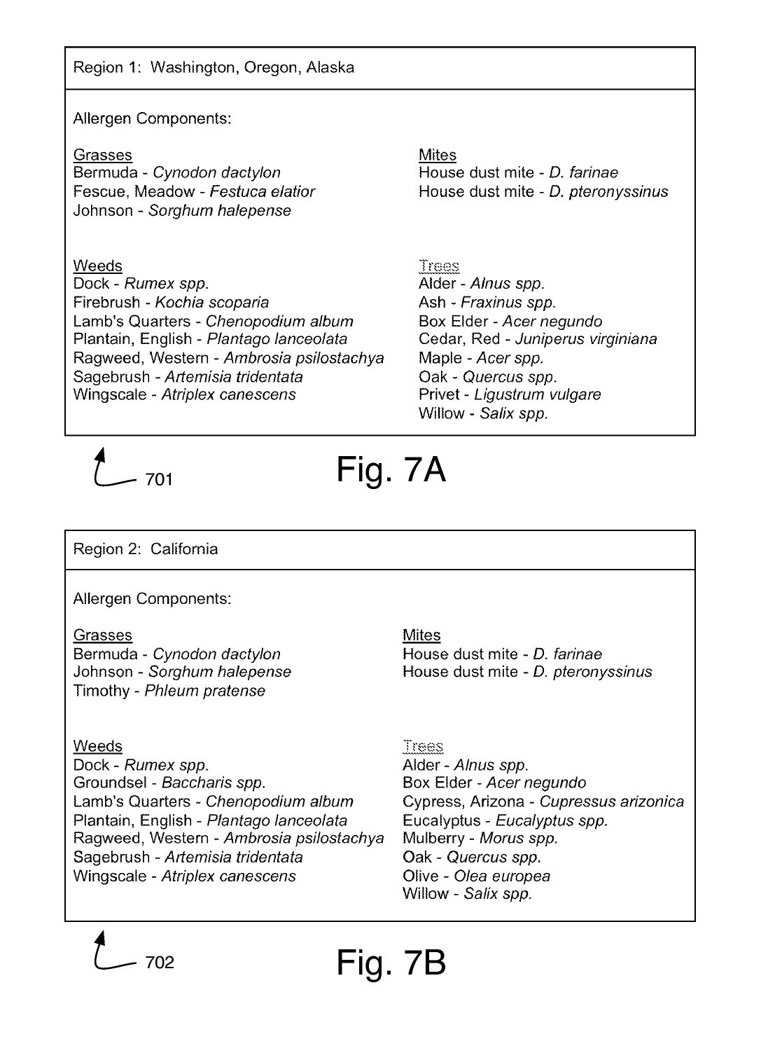

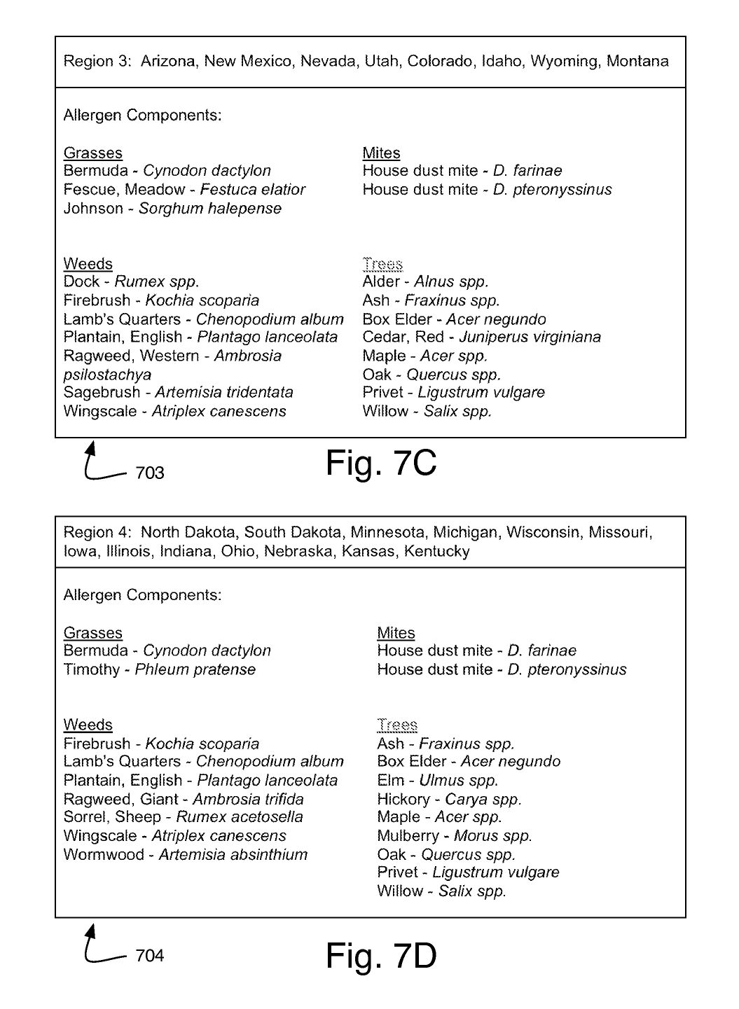

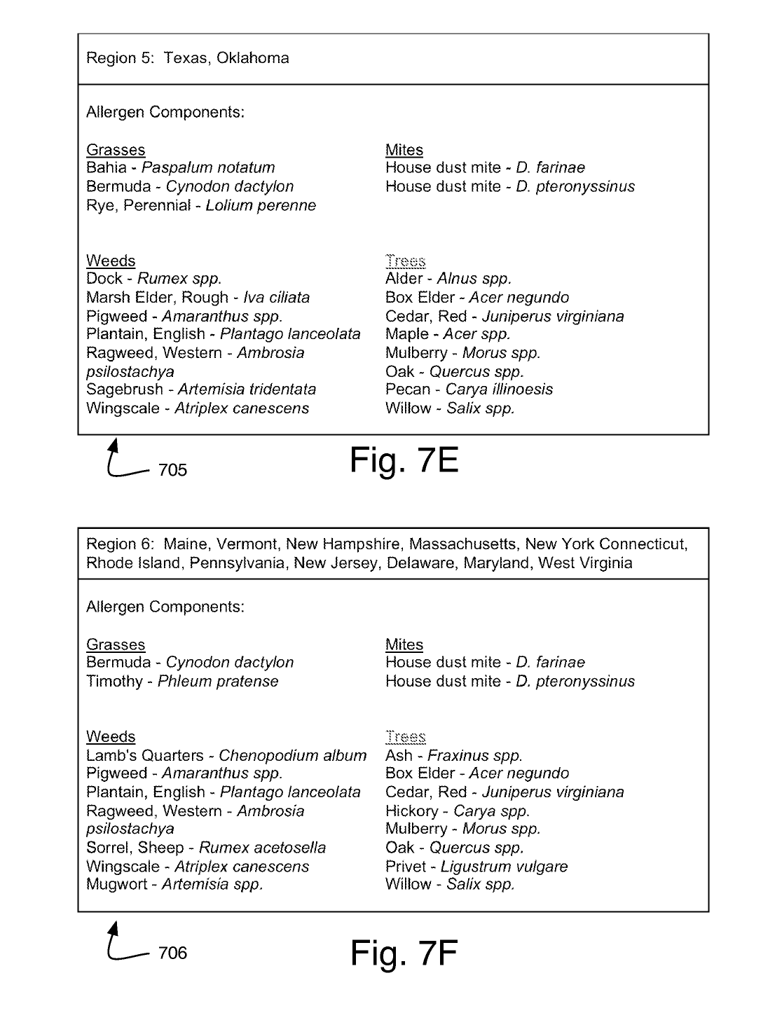

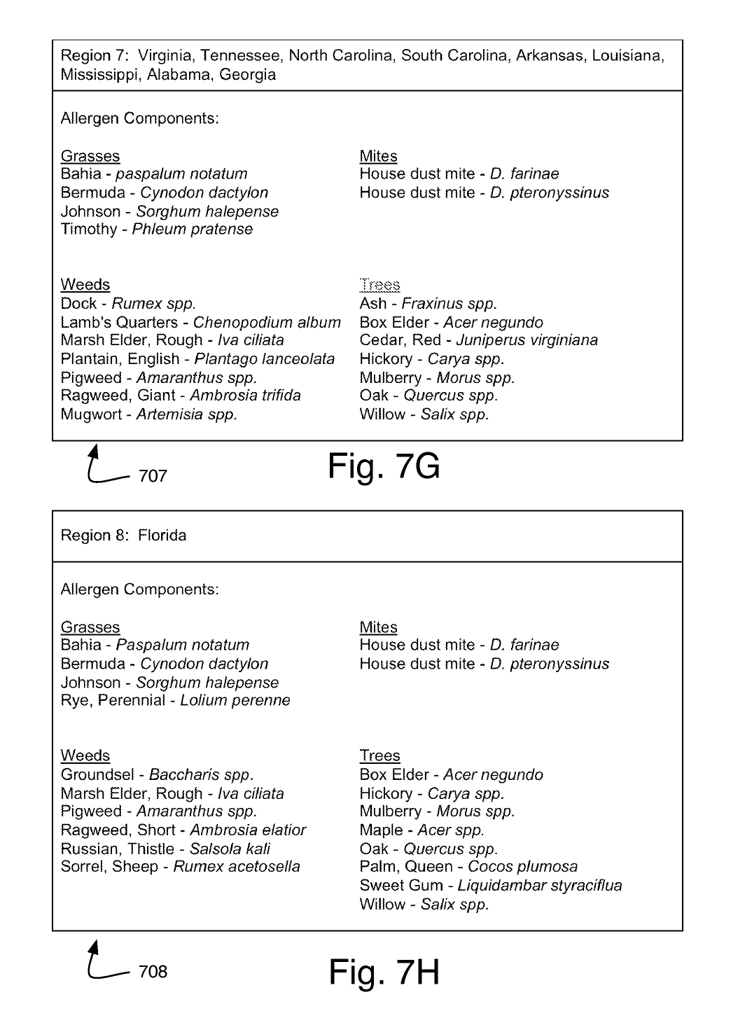

Reference is now made to FIG. 6, which illustrates an example group of geographic regions. As shown in FIG. 6, in an example, the geographic region associated with the companion animal may be one of several geographic regions of the United States. In an example, the United States may be divided into about eight geographic regions 1, 2, 3, 4, 5, 6, 7 and 8. The states associated with each example geographic region are summarized in Tables 701, 702, 703, 704, 705, 706, 707 and 708 of FIGS. 7A to 7H. As will be appreciated, FIG. 6 is an example of a group of regions that the area of the United States may be divided into such that a companion animal may be associated with a region for the determination of a formulation for allergy immunotherapy. As is discussed further herein, in general, any geographic area may be divided into any number and variety of geographic regions such that a companion animal may be associated with the region for the determination of an allergy immunotherapy mixture formulation.

For the sake of an illustrative example, it may be assumed that a veterinarian in Portland, Oreg. has diagnosed atopic dermatitis in a dog, and, after evaluation, the veterinarian has concluded that the dog lives in Portland, Oreg. In the illustrative example, the location associated with the companion animal may be Portland, Oreg. and, with reference to FIG. 6, the geographic region associated with the companion animal dog may be Region 1.

Returning now to FIG. 1, method 100 may continue at block 140. At block 140, an allergy immunotherapy formulation may be determined. In general, the allergen immunotherapy formulation may define the components of an allergy immunotherapy mixture that may be procured and administered to a companion animal. In an example, each geographic region may have an associated allergen immunotherapy formulation. In various examples, the association may be made by a veterinarian, a veterinarian's assistant or the like, a third party immunotherapy specialist, or an allergen manufacturer, or the like. In some examples, the association may be made by reference to a map, a data sheet, a print publication, a print advertisement, a catalogue, a digital publication, a publication on an Internet web site, or the like.

Again with reference to FIG. 6, and as discussed, in an example, the United States may be divided into geographic regions 1 to 8. In the illustrated example, each geographic region may have an associated allergen immunotherapy formulation which may be summarized in Tables 701, 702, 703, 704, 705, 706, 707 and 708 of FIGS. 7A to 7H. In the illustrative example discussed above, a dog diagnosed with atopic dermatitis and associated with Region 1 may be ordered to be administered with the formulation as summarized in Table 701 shown in FIG. 7A. Method 100 may continue at block 150.

At block 150, an allergy immunotherapy mixture may be generated based on the allergy immunotherapy formulation. In general, the allergy immunotherapy mixture may include allergen components defined by the allergy immunotherapy formulation. In an example, the mixture may include the allergen components provided in a suitable aqueous solution for administration to the companion animal. In an example, the allergy immunotherapy mixture may be produced by a manufacturer. In another example, the components of an allergy immunotherapy mixture may be procured and combined by a veterinarian, a veterinarian's assistant, an allergy immunotherapy specialist, or the like. The method may continue at block 160.

At block 160, the mixture may be administered to the companion animal. In various examples, the administration may be performed by a veterinarian, a veterinarian's assistant or the like, a third party immunotherapy specialist, or a companion animal's owner. In general, the administration may follow standard veterinary protocols under the general supervision of a veterinarian. In some examples, the administration may be by injection. In other examples, the administration may be oral.

In general, method 100 may be repeated for another companion animal. In such examples, the companion animal may be from the same or a different geographical region. It will be appreciated that method 100 may be repeated any number of times and that the herein discussed methods provide a general methodology for the determination and acquisition of an allergen mixture for companion animals in any of the described geographic regions.

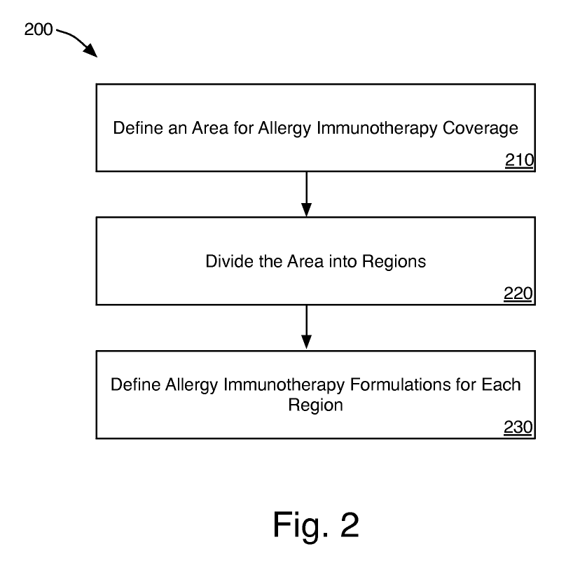

FIG. 2 illustrates a flow chart of an example method for generating regions and allergy immunotherapy formulations. In general, some or all of the functions described with respect to blocs 210 to 230 may be performed by a veterinarian, an allergy immunotherapy specialist, an allergy immunotherapy manufacturer, or the like. Method 200 may begin at block 210.

At block 210, an area for the coverage of allergy immunotherapy in companion animals may be defined. In general, the area may be any suitable geographic area for which formulation coverage may be desired. With reference to FIG. 6, in an example, the area may be the United States. In general, the area may be any geographic area for which allergy immunotherapy formulation coverage may be desired. The method may continue at block 220.

At block 220, the area may be divided into regions. In general, the area may be divided into any number of suitable regions such that the allergy immunotherapy formulations for each of the regions have a suitable efficacy. With reference to FIG. 6, in an example, the area of the United States may be divided into regions along state borders. Such an implementation may offer the advantage of simplicity. However, many region divisions may be available as is discussed further herein. The method may continue at block 230.

At block 230, allergy immunotherapy formulations may be defined for each of the regions established at block 220. As discussed, FIG. 6 is an illustration of the area of the United States divided into eight geographic regions 1, 2, 3, 4, 5, 6, 7 and 8. FIGS. 7A to 7H are tables illustrating the states in the regions of FIG. 6 and corresponding formulations of allergen components in this example. As will be appreciated, FIGS. 6 and 7A to 7H illustrate an example implementation of the methods discussed herein. In the illustrated example, the area of the United States has been divided into eight regions that include one or more states. Such an example may offer the advantages of a balance between the size of the regions, the number of regions, the efficacy of the region specific allergen immunology mixtures and simplicity of implementation.

In general, any area may be divided into any number of geographic regions such that a companion animal may be associated with the region for the determination of an allergy immunotherapy mixture formulation. In some examples, different states of the United States may be combined into different geographic regions. In various examples, the United States may be divided into two to twenty regions. In other examples, the regions may not be based on the borders of states but instead may be based on a shared geography, biogeography, climate, biome, ecosystem, pollination zone or other characteristic or characteristics. In an example, the continental United States may be divided into dry, humid temperate and humid tropical regions according to the Bailey System of biogeographical classification. Many other applicable divisions may be available.

In the illustrated example, the area being divided into regions is generally the United States. In other examples, the area divided may be an area other than the United States, such as, for example, a portion of the United States, any country, any group of countries, a continent, or a portion of a continent, or the like. Generally, an area may be divided into geographic regions and, as discussed, each geographic region may correspond to a mixture of allergen components that may be administered to companion animals associated with that region. For example, companion animals in Region 1 may be administered the Region 1 mixture, which is summarized in table 701 of FIG. 7A. Similarly, in the illustrated example, companion animals in Regions 2, 3, 4, 5, 6, 7 and 8 may be administered the mixtures summarized in tables 702, 703, 704, 705, 706, 707 and 708, respectively (as illustrated in FIGS. 7B, 7C, 7D, 7E, 7F and 7G). In general, any allergen component formulations may be used that have sufficient efficacy to alleviate symptoms in the treated companion animal.

In the illustrated example, each type of companion animal (e.g., dog, cat or horse) may use the same regions and allergy immunotherapy formulations. In other examples, the regions and formulations may be companion animal specific. For example, the regions and formulations for dogs, cats and horses may be different.

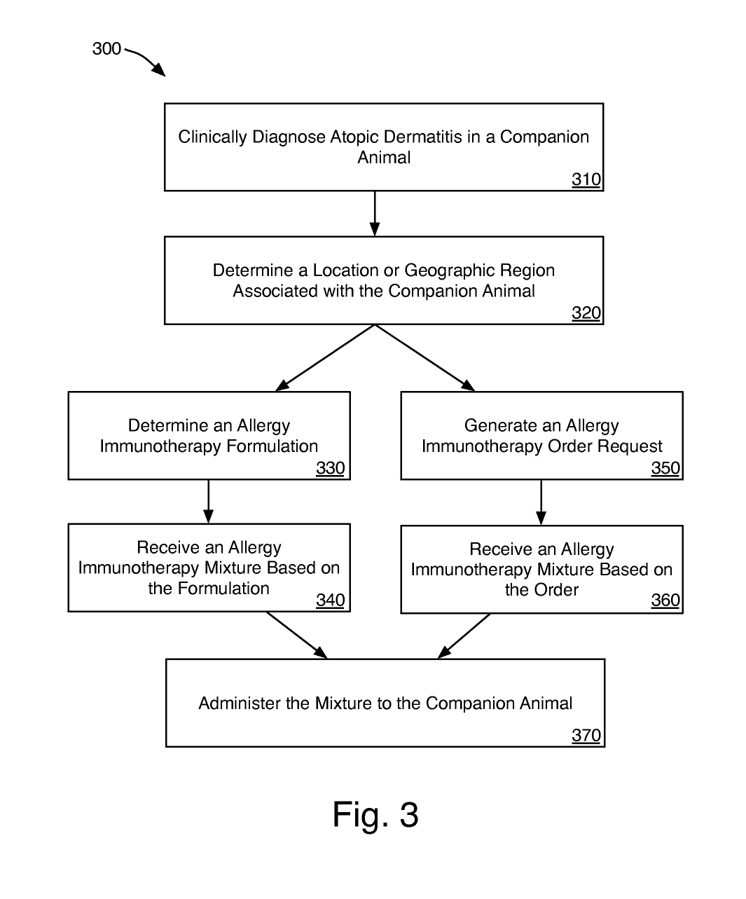

FIG. 3 illustrates a flow chart of an example method for providing allergy immunotherapy to a companion animal. In general, some or all of the functions described with respect to blocks 310 to 370 may be performed by a veterinarian, by staff at a veterinary clinic or by equipment or computing devices at a veterinary clinic. Method 300 may commence at block 310. At block 310, a veterinarian may diagnose atopic dermatitis in a companion animal. Any of the techniques described herein may be used for the diagnosis. The method may continue at block 320.

At block 320, a location or geographic region may be associated with the diagnosed companion animal. In general, the location or geographic region may be a location indicator. The location indicator may be associated with the companion animal and may define either a location of the animal or the geographic region of the animal. Any of the techniques described herein may be used to determine the associated location or geographic region. In an example, a location may include a description of a physical location associated of the animal. In an example, a geographic region may include a geographic region that may be used to determine a allergen formulation for the animal. With reference to FIG. 6, in some examples, a location may generally provide a location on a map while a geographic region may describe at least one of regions 1, 2, 3, 4, 5, 6, 7 or 8. In the illustrative example where an animal that lives in Portland, Oreg. is diagnosed, the associated location may be Portland, Oreg. (or, e.g., Oregon, a street address in Portland, Oreg., or a zip code or the like), and the associated geographic region may be Region 1, and the location indicator may include information related to the location or geographic region. The method may continue at block 330 or block 350.

In some examples, the veterinarian or the veterinary clinic staff may determine an allergy immunotherapy formulation (as illustrated at block 330). In such examples, the allergy immunotherapy formulation may be determined based on an allergy immunotherapy expert's publication or a manufacturer's publication. Such publications may be supplied via any communication medium, such as, for example, print publications, print advertisements, catalogues, digital publications, e-mails, publication on an Internet web site, or the like. The veterinarian (or the clinic's staff) may consult a publication to determine an allergy immunotherapy formulation. The method may continue at block 340. At block 340, in such examples, the veterinarian or clinic may place an order and receive an allergy immunotherapy mixture corresponding to the allergy immunotherapy formulation. In an example, the order may be received from a manufacturer. In another example, the order may be placed with an allergy immunotherapy specialist, and the mixture may be received either from the specialist or directly from a manufacture (e.g., based on an order placed by the specialist). The method may continue at block 370.

At block 370, the mixture may be administered to the companion animal. Any of the techniques discussed herein may be used for application of the mixture to the companion animal.

In other examples, the veterinarian or the veterinary clinic staff may generate an allergy immunotherapy order request that may include the location or geographic region of the companion animal (as illustrated by block 350). In some examples, the order request may include the type of animal to be treated. In general, the order may also include other information common in sales transactions. In some examples, the order request may be transmitted to an allergy immunotherapy specialist. In other examples, the order request may be transmitted to a manufacturer. The order may be transmitted in any suitable fashion including, for example, by mail, by email, by submission over an Internet web site, or the like. The method may continue at block 360. At block 360, in such examples, the veterinarian or clinic may receive an allergy immunotherapy mixture corresponding to the order request (the allergy immunotherapy or manufacturer may provide the service of determining the associated formulation for providing the allergy immunotherapy mixture). The method may continue at block 370, which may include the techniques as described above.

In general, method 300 may be repeated for any number of companion animals, which may be from the same or different locations or the same or different geographies. It will be appreciated that method 300 may be repeated any number of times and that the method provides a general methodology for the determination and acquisition of an allergen mixture for companion animals in any of the described geographic regions.

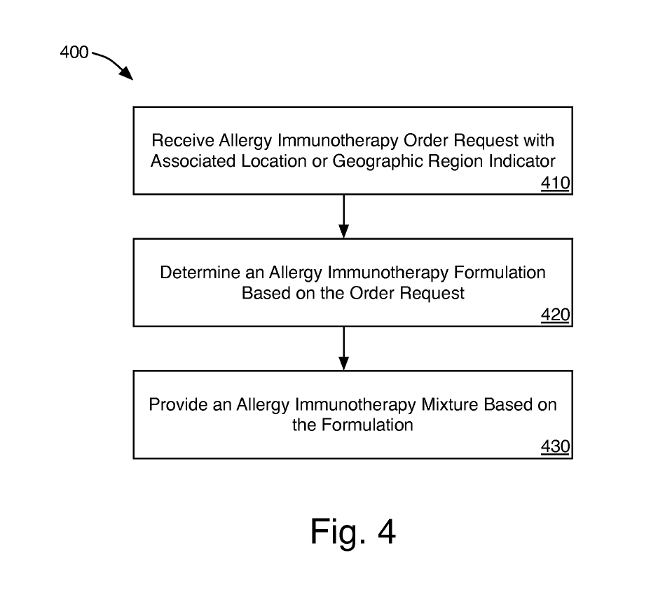

FIG. 4 illustrates a flow chart of an example method for providing allergy immunotherapy to a companion animal. In general, some or all of the functions described with respect to blocks 410 to 430 may be performed by an allergy immunotherapy specialist, by staff of an allergy immunotherapy specialist, or by equipment or computing devices of the allergy immunotherapy specialist. Method 400 may begin at block 410. At block 410, an order request may be received. The order request may include a location or a geographic region indicator associated with the companion animal for which treatment is sought. In general, the location or geographic region may be a location indicator. The location indicator may be associated with the companion animal and may define either a location of the animal or the geographic region of the animal. As discussed, in some examples, the location may include an address or the like for the companion animal and a geographic region indicator may include a selected one of a plurality of geographic regions that is associated with the companion animal's location. The order request may be received by any suitable technique, such as, for example, by mail, by phone, by email, by submission of a form through an Internet website, or the like. The method may continue at block 420.

At block 420, an allergy immunotherapy formulation based on the order request may be determined. In examples where an location indicator associated with the companion animal is received, a geographic region may be determined by any technique as discussed herein. Based in part on the determined geographic region, an allergy immunotherapy formulation may be determined for eventual administration to the companion animal. In other examples, the order request may include the geographic region associated with the companion animal. In such examples, the allergy immunotherapy specialist (or their staff) may determine an allergy immunotherapy formulation based on the animal's geographic region. The method may continue at block 430.

At block 430, the allergy immunotherapy mixture may be provided. In some examples, the allergy immunotherapy specialist may prepare the allergy immunotherapy mixture and send the mixture to a veterinarian for administration to the companion animal. In other examples, the allergy immunotherapy specialist may provide the allergy immunotherapy formulation as an order to a allergy immunotherapy mixture manufacturer such that the mixture may be produced and sent to the veterinarian (either through the specialist or directly from the manufacturer).

In general, method 400 may be repeated for any number of companion animals, which may be from the same or different locations or the same or different geographies. It will be appreciated that method 400 may be repeated any number of times and that the method provides a general methodology for the determination and acquisition of an allergen mixture for companion animals in any of the described geographic regions.

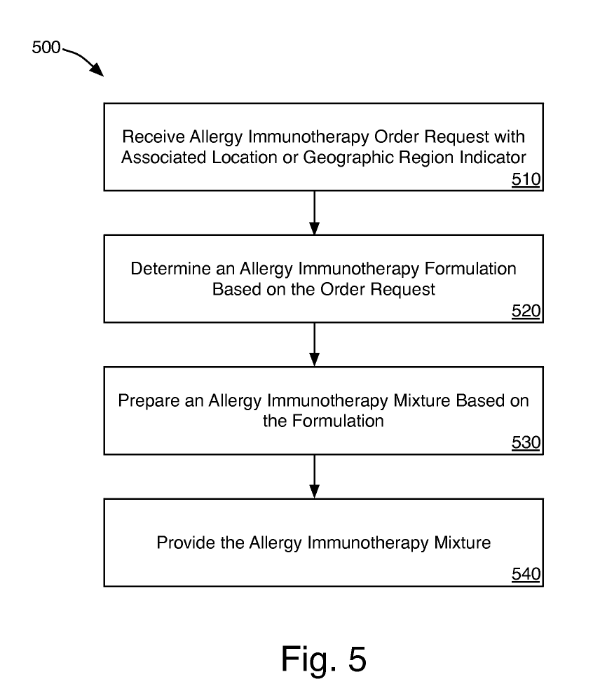

FIG. 5 illustrates a flow chart of an example method for providing allergy immunotherapy to a companion animal. Method 500 may include functional blocks 510-540. In general, some or all of the functions described with respect to blocks 510-540 may be performed by an allergy immunotherapy manufacturer or by equipment or computing devices of the allergy immunotherapy manufacturer. The method may start at block 510. At block 510, an order request may be received. The order request may include a location or a geographic region indicator associated with a companion animal being treated for atopic dermatitis. As discussed, in some examples, the location may include an address or the like for the companion animal and a geographic region indicator may include a selected one of a plurality of geographic regions that is associated with the companion animal's location. The order request may be received by any suitable technique, such as, for example, by mail, by phone, by email, by submission of a form through an Internet website, or the like. The method may continue at block 520.

At block 520, an allergy immunotherapy formulation based on the order request may be determined. In examples where an associated with the companion animal is received, a geographic region may be determined by any technique as discussed herein. Based in part on the determined geographic region, an allergy immunotherapy formulation may be determined for eventual administration to the companion animal. In other examples, the order request may include the geographic region associated with the companion animal. In such examples, the allergy immunotherapy manufacturer may determine an allergy immunotherapy formulation based on the animal's geographic region. In other examples, the manufacture may receive an order request that includes the formulation. In such examples, manufacturer may skip functional block 520. The method may continue at block 530.

At block 530, the manufacturer may prepare the allergy immunotherapy mixture by standard techniques. In some examples, the manufacturer may combine allergen components into a mixture. In other examples, the manufacturer may package the allergen components such that they may be mixed at a later time. The method may continue at block 540. At block 540, the allergy immunotherapy mixture may be provided. The mixture may be delivered to a client veterinarian for administration to a companion animal.

In general, method 500 may be repeated for any number of companion animals, which may be from the same or different locations or the same or different geographies. It will be appreciated that method 500 may be repeated any number of times and that the method provides a general methodology for the determination and acquisition of an allergen mixture for companion animals in any of the described geographic regions.

While there has been illustrated and/or described what are presently considered to be example embodiments of claimed subject matter, it will be understood by those skilled in the art that various other modifications may be made, and/or equivalents may be substituted, without departing from the true scope of claimed subject matter. Additionally, many modifications may be made to adapt a particular situation to the teachings of claimed subject matter without departing from subject matter that is claimed. Therefore, it is intended that the patent not be limited to the particular embodiments disclosed, but that it covers all embodiments falling within the scope of the appended claims.

What is claimed is:

1. A method for providing allergy immunotherapy to a companion animal comprising:
receiving a first companion animal allergy immunotherapy order request including a first location indicator associated with a first geographic region;
receiving a second companion animal allergy immunotherapy order request including a second location indicator associated with a second geographic region;
determining a first allergy immunotherapy formulation based at least in part on the first geographic region;
determining a second allergy immunotherapy formulation based at least in part on the second geographic region, wherein the first allergy immunotherapy formulation and the second allergy immunotherapy formulation have different allergy immunotherapy formulations; and
providing the first allergy immunotherapy formulation for fulfillment of a mixture order.

2. The method of claim 1, further comprising:
fulfilling the mixture order by sending an immunotherapy mixture based at least in part on the first allergy immunotherapy formulation to a client.

3. The method of claim 1, wherein the first allergy immunotherapy formulation and the second allergy immunotherapy formulation contain no common components.

4. The method of claim 1, wherein the first allergy immunotherapy formulation and the second allergy immunotherapy both include a common component.

5. The method of claim 1, wherein the first geographic region comprises one or more states of the United States.

6. The method of claim 1, wherein the first geographic region comprises a region defined by at least one of a biogeography, a climate, a biome, a pollination zone or an ecosystem.

7. A method for providing allergy immunotherapy to a companion animal comprising:
determining a first allergy immunotherapy formulation based at least in part on a first geographic region associated with a first companion animal;
determining a second allergy immunotherapy formulation based at least in part on a second geographic region associated with a second companion animal, wherein the first allergy immunotherapy formulation and the second allergy immunotherapy formulation have different formulations;
ordering an allergy immunotherapy mixture based at least in part on the first allergy immunotherapy formulation; and
administering the allergy immunotherapy mixture to the first companion animal.

8. The method of claim 7, wherein determining the first allergy immunotherapy formulation based at least in part on the first geographic region comprises consulting at least one of a print publication, a print advertisement, a catalogue, a digital publications or a publication on an Internet web site.

9. The method of claim 7, further comprising:
diagnosing atopic dermatitis in the companion animal.

10. The method of claim 7, wherein the first allergy immunotherapy formulation and the second allergy immunotherapy both include a common component.

11. The method of claim 7, wherein the first geographic region comprises one or more states of the United States.

12. A method for providing allergy immunotherapy to a companion animal comprising:
receiving a first companion animal allergy immunotherapy order request including a first location indicator associated with a first geographic region;
receiving a second companion animal allergy immunotherapy order request including a second location indicator associated with a second geographic region;
determining a first allergy immunotherapy formulation based at least in part on the first geographic region;
determining a second allergy immunotherapy formulation based at least in part on the second geographic region, wherein the first allergy immunotherapy formulation and the second allergy immunotherapy formulation have different formulations; and
providing a mixture based at least in part on the first allergy immunotherapy formulation for administration to the first companion animal.

13. The method of claim 12, wherein the first location indicator identifies the first geographic region.

14. The method of claim 12, wherein the first location indicator indentifies a location is in the first geographic region.

15. The method of claim 12, wherein determining the first allergy immunotherapy formulation based at least in part on the first geographic region comprises consulting at least one of a print publication, a print advertisement, a catalogue, a digital publications or a publication on an Internet web site.

16. The method of claim 12, wherein the first allergy immunotherapy formulation and the second allergy immunotherapy formulation contain no common components.

17. The method of claim 12, wherein the first allergy immunotherapy formulation and the second allergy immunotherapy both include a common component.

18. The method of claim 12, wherein the first geographic region comprises one or more of the United States.

19. The method of claim 12, wherein the first geographic region comprises a region defined by at least one of a biogeography, a climate, a biome, pollination zone or an ecosystem.

20. The method of claim 12, wherein the first companion animal comprises at least one of a dog, a cat or a horse.
